(19) United States
(12) Patent Application Publication
(10) Pub. No.: US 2013/0225951 A1
(43) Pub. Date: Aug. 29, 2013

(54) DETERMINATION OF OXYGEN SATURATION IN A TISSUE OF VISUAL SYSTEM

(71) Applicants: Bahram Khoobehi, Metairie, LA (US); Hussein Wafapoor, Naples, FL (US); Alexander M. Eaton, Fort Meyers, FL (US); Paul Fournier, Albuquerque, NM (US)

(72) Inventors: Bahram Khoobehi, Metairie, LA (US); Hussein Wafapoor, Naples, FL (US); Alexander M. Eaton, Fort Meyers, FL (US); Paul Fournier, Albuquerque, NM (US)

(73) Assignee: EYE02 SCAN LLC, Fort Meyers, FL (US)

(21) Appl. No.: 13/643,956

(22) PCT Filed: Apr. 26, 2011
(86) PCT No.: PCT/US11/33939

§ 371 Date: Dec. 18, 2012

Related U.S. Application Data
(60) Provisional application No. 61/329,205, filed on Apr. 29, 2010.
(60) Provisional application No. 61/478,847, filed on Apr. 25, 2011.

(51) Int. Cl. A61B 5/1455

(52) U.S. Cl. CPC A61B 5/14555
USPC 600/318; 600/310

(57) ABSTRACT

A method and system of acquisition and processing of data representing oxygen saturation (OS) value of a tissue of a visual system of a subject, such as the optic nerve head and overlying artery and vein. The data is acquired at pre-determined discrete spectral points, including at least two isosbestic points, as a discrete reflectance spectrum, with the use of a multi-spectral optical imaging system that simultaneously produces a plurality of two-dimensional spectrally-discrete images by segmenting an incoming light front with secondary objectives. The OS value is assessed based on determination of areas bound by an acquired discrete reflectance spectrum and an isosbestic line.

### CROSS-REFERENCE TO RELATED APPLICATIONS

The present application claims priority from U.S. Provisional Patent Application No. 61/329,205 titled “Single Exposure Multispectral Camera” and filed on Apr. 29, 2010, and U.S. Provisional Patent Application No. 61/478,847 titled “Determination of Oxygen Saturation in a Tissue of Visual System” and filed on Apr. 25, 2011. The disclosure of each of the above-mentioned applications is incorporated herein in its entirety by reference.

### TECHNICAL FIELD

This invention pertains broadly to monitoring changes of pathologic conditions in the retina and optic nerve head and, in particular, to use of simultaneous acquisition of reflection characteristics of the retinal scene at pre-determined discrete wavelengths to characterize the relative spatial changes in retinal oxygen saturation.

### BACKGROUND

The visual system, as part of the central nervous system enables an organism to process visual information. It interprets information from visible light to build a representation of the surrounding world. The visual system accomplishes a number of complex tasks, including the reception of light and the formation of monocular representations; the construction of a binocular perception from a pair of two dimensional projections; the identification and categorization of visual objects; assessing distances to and between objects; and guiding body movements in relation to visual objects. Quite understandably, the health of the visual system is of critical importance for keeping the organism to be efficiently operational.

Pathologic conditions in the retina and optic nerve head (ONH) can cause vision loss and blindness. Both structures have a high demand for oxygen, and loss of the normal oxygen supply through vascular insufficiency is believed to play an important role in diseases affecting the retina and ONH. Hypoxia of the retina and ONH is believed to be a factor in the development of ocular vascular disorders, such as diabetic retinopathy, arteriovenous occlusion, and glaucoma. The ability to obtain relative measurements of oxygen saturation in the human ocular fundus could aid diagnosis and monitoring of these and other disorders. For example, measurement of changes in retinal and ONH oxygen saturation under controlled conditions could establish relationships among oxygen consumption, blood sugar levels, and vascular autoregulatory function in diabetic retinopathy. Moreover, the assessment of oxygenation in the ONH may facilitate early detection of the onset of glaucoma, a disease in which timely diagnosis is crucial for effective treatment.

Several attempts to develop a methodology of accurate assessment of oxygen content such as a level of oxygen saturation (OS) in a human visual system were discussed in related art and include, among others, physically-invasive techniques, the use of phosphorescent dyes in an eye of a human subject (which has not been approved yet), and techniques based on evaluation of reflectance of a component of the visual system. One of the biggest obstacles of using imaging to acquire information relevant to OS remain short times of saccadic movements of the eye that are generally insufficiently long for sequential collection of spectral information about the eye.

### SUMMARY OF THE INVENTION

Embodiments of the present invention prove a method for determining an oxygen saturation signature of an ocular tissue or a component of an eye such as, for example, retina blood vessels, retinal tissue, optic nerve head and vessels, choroid, and iris. The method includes receiving optical data representing a spectral distribution of light that has been reflected by multiple points of the ocular tissue and that is defined by a predetermined number of discrete wavelengths at least two of which are isosbestic wavelengths. In a specific embodiment, the received optical data may represent a distribution of reflected light acquired during a time period that is shorter than a duration of a saccade. The method further includes processing the received optical data for each point of the ocular tissue such as to determine first and second spectral distribution lines on a spectral graph, determine a first area of spectral graph regions that are bound by these first and second spectral distribution lines, and normalizing this determined value of the area by a value of a second area under the second spectral distribution line. In a specific embodiment, the second area may be independent from a level of oxygen saturation on the ocular tissue at a given point at the tissue. In one embodiment, determination of the first area may include determination of the area of three spectral graph regions that are bound by the first and second spectral distribution lines and that adjoin each other at isosbestic points. The method may further include assigning thus determined normalized value to a corresponding point of the ocular tissue and storing this assigned value in an array representing a two-dimensional distribution of points across a region of interest of the ocular tissue, and storing these assigned values. In addition, the method may contain at least one of the steps of acquiring the optical data and presenting the assigned normalized values as a map of 2D distribution of the oxygen saturation signature of the ocular tissue across the region of interest. In a specific embodiment, such map may include a color-coded image of the region of the ocular tissue, where the color-coding represents levels of oxygen saturation values.

Embodiments of the invention also provide for a computer program product encoded in a computer-readable medium and usable with a programmable computer processor disposed in a computer system. According to the idea of the invention, the computer program product includes a computer-readable program code which causes the computer processor to receive data, from an optical detector, that represent a discrete spectral distribution of intensity of light reflected by a fundus of a subject and detected at predetermined wavelengths at least two of which are isosbestic wavelengths. In a specific embodiment, the received data represents a discrete spectral distribution of light reflected from a fundus and detected within a period of time that is no longer that a duration of a saccadic movement of the eye of the subject. In addition, the computer program product includes a computer-readable program code that causes the processor to transform the received data into data representing oxygen saturation of blood in the retina. The transformation of the received data may include normalization of the oxygen saturation level with respect to at least one of the amount of blood in a portion of fundus, that has been imaged, and the intensity of light that has been detected. The processor may also be caused to display a color-coded map of spatial distribution of the oxygen saturation values across the imaged portion of fundus.

Embodiments of the invention additionally provide for a computer program product for displaying a color-coded map of oxygen saturation levels corresponding to a component of an eye. The component of an eye may include a retina, an optical nerve head, a choroid, an iris, or any other ocular tissue. Such computer program product includes a computer-readable tangible and non-transitory medium having a computer-readable program code thereon which includes a program code for acquiring digital data representing intensity of light that has interacted with the component of an eye and that has been detected with at least one optical detector at a plurality of discrete wavelengths at least two of which are isosbestic wavelengths. In a specific embodiment the data is acquired in a time period that is shorter than a duration of a saccadic movement of the eye. The computer program product may also include program code for determining a first curve that represents, in a chosen system of coordinates, a distribution of intensity values associated with the acquired digital data as a function of the plurality of discrete wavelengths, and program code for determining a second curve representing, in the same system of coordinates, a distribution of isosbestic intensity values as a function of the isosbestic wavelengths. The transformation of the received data may include normalization of the oxygen saturation level with respect to at least one of the amount of blood in a portion of the component of an eye that has been imaged and the intensity of light that has been detected. In addition, the computer program product may include program code for deriving, in the system of coordinates, values that represent area bound by the first and second curves and that correspond to the oxygen saturation levels. Additional program code may be used for storing the received data, the isosbestic data, and the data defining the second and first curves, and, additionally or alternatively, for assigning a color parameter for each of data points from the acquired digital data based on intensity values respectively corresponding to these data points.

### BRIEF DESCRIPTION OF THE DRAWINGS

The foregoing features of the invention will be more readily understood by reference to the following detailed description, taken with reference to the accompanying, drawn not-to-scale, figures where like features and elements are denoted by like numbers and labels, and in which:

FIG. 1 schematically shows an embodiment of the present invention;

FIG. 2 illustrates a single image frame and a resulting composite multi-spectral image produced with the use of embodiment of FIG. 1;

FIG. 3 schematically shows an exemplary the use of another embodiment of the invention use in retinal imaging;

FIG. 4 depicts an optical layout of the embodiment of FIG. 3;

FIG. 5 illustrates, in perspective view, a re-imaging sub-system of the layout of FIG. 4;

FIG. 6 shows an image frame and the resulting composite multi-spectral image produced with the use of the embodiment of FIGS. 3 and 4;

FIG. 7 illustrates means of axial repositioning of secondary objective of the re-imaging sub-system of the embodiment of FIGS. 3 and 4.

FIG. 8 is a layout of an alternative embodiment of the invention;

FIGS. 9 provides an example of practical integration of an embodiment having the layout of FIG. 8 with an auxiliary imaging device; and

FIG. 10 shows an image frame containing individual spectrally-separate images obtained with the use of the integrated system of FIG. 9.

FIG. 11 summarizes optical design parameters of the embodiment of FIG. 8.

FIG. 12 shows a spot-diagram of an optical system similar to that of FIG. 8 but without segmentation of the exit pupil of the relay-subsystem with an array of optical lenses.

FIG. 13 shows a spot-diagram corresponding to the embodiment of FIG. 8.

FIG. 14 illustrates transmission characteristics of optical filters employed in the embodiment of FIG. 8.

FIGS. 15 through 17 show fundus retinal images, obtained at different times, of a patient suffering from central retinal artery occlusion.

FIGS. 18 and 19 present analyzed images corresponding to images of FIGS. 15 through 19.

FIG. 20 depicts a discrete reflectance spectrum of a retinal artery acquired according to an embodiment of the invention.

FIG. 21 shows the spectrum of FIG. 20 and a corresponding isosbestic line.

FIGS. 22A, 22B illustrate graphical determination of an OS value according to an embodiment of the invention.

FIG. 22C illustrates an isosbestic line of FIG. 21 overlapped with a continuous reflectance spectrum of the retina.

FIG. 23 provides a flow-chart schematically illustrating an embodiment of an algorithm according to a method of the invention.

FIG. 24 depicts an optical image of retina obtained with a multi-spectral camera of the invention.

FIG. 25 illustrates discrete reflectance spectra of blood in veins and arteries of an eye, obtained as readout from 16-bit CCD camera.

FIGS. 26A, 26B, 26C, 26D, 26E show color-coded 2D distributions of an OS characteristic across the portion of retina derived from data acquired and processed according to an embodiment of the invention.

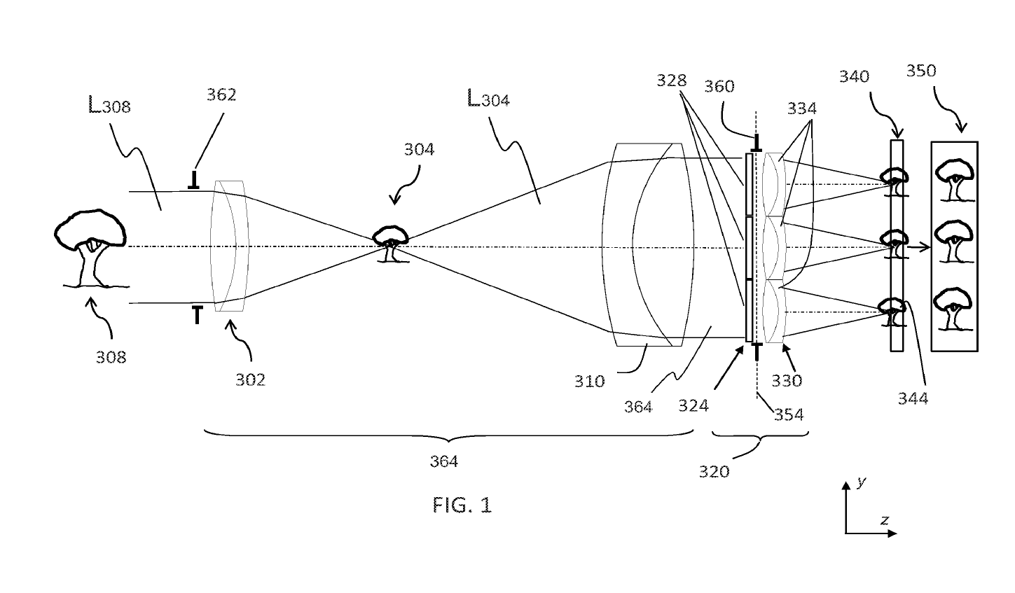

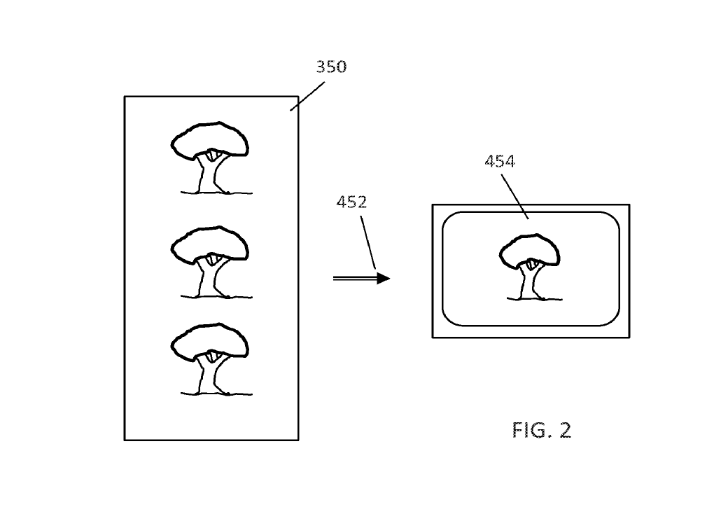

### DETAILED DESCRIPTION OF SPECIFIC EMBODIMENTS

For the purpose of the application and the appended claim, the following terms are defined as described unless the context requires otherwise. The term “image” refers to an ordered representation of detector signals corresponding to spatial positions. For example, an image may be an array of values within an electronic memory, or, alternatively, a visual image may be formed on a display device X such as a video screen or printer.

The following specification provides a description of the embodiments of the invention with reference to the accompanying drawings. In the drawings, wherever possible, the same reference numerals and labels refer to the same or like components or elements. It will be understood, however, that similar components or elements may also be referred to with different numerals and labels.

Throughout this specification, a reference to “one embodiment,” “an embodiment,” or similar language implies that a particular feature, structure, or characteristic described in connection with the embodiment referred to is included in at least one embodiment of the present invention. Thus, phrases “in one embodiment,” “in an embodiment,” and similar terms used throughout this specification may, but do not necessarily, all refer to the same embodiment. Moreover, it will be understood that features, elements, components, structures, details, or characteristics of various embodiments of the invention described in the specification may be combined in any suitable manner in one or more embodiments. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention.

The schematic flow chart diagram that is included is generally set forth as a logical flow-chart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Collection of information about oxygenation levels in the human visual system have been previously carried out with various methods including, but not limited to, physically invasive measurements of oxygen tension (Po₂) in the ONH using O₂-sensitive microelectrodes inserted into the eye; injection of a phosphorescent dye has been used to study Po₂ in the retinal and choroidal vessels, as well as the microvasculature of the ONH rim; and spectral imaging. While the first methodology allows to relatively accurately determine Po₂ distribution in three dimensions, its invasive nature limits its use to animal models and precludes clinical application. The second technique has not been approved for use in humans yet. Spectral imaging, on the other hand, is a non-invasive technique that can be a powerful tool for identifying retinal hypoxia that is associated with established stages of diabetic retinopathy (DR), for example. Results of several oximetry studies conducted with the use of spectral imaging indicate that the interest in developing methodology of oxymetry and its applications to studies of retinal disorders is increasing.

Conventional hyperspectral imaging approach, when used for detecting optical spectra of a human eye in reflection, for example, employs sequential one-dimensional imaging across a chosen spectral region (such as the entire visible and infra-red, IR, region) with a chosen spectral resolution. The technique uses a push-broom style scanner. Each acquired imaging frame holds the spatial (x) and spectral (λ) axis for each line of the acquired hyper-spectral image, with successive lines of the frame forming the z-axis in the stack of frames. A “band-sequential” hyper-spectral image is obtained by rotation of the stack of images, interchanging the z and λ axes. After rotation, each frame contains a two-dimensional spatial image at a distinct wavelength in which intact structures are recognizable. A push-broom scanner spectrometer is a scanning spectral imaging device conventionally used for image acquisition. The scanner employs a lens system to focus input light (such as light reflected from fundus) onto a field-limiting entrance slit and collimate the light. The collimated light is then spectrally dispersed (by, e.g., a diffraction grating) and focused on a CCD camera to acquire spectral information. The device allows a two-dimensional (2D) sensor detector to sample the spectral dimension and one spatial dimension (e.g., x-axis) simultaneously. As a result, a one-dimensional (1D), line spectral image is obtained. Information in another spatial dimension (e.g., z-axis) is generated by spatial scanning. The resulting full image is obtained by appropriate compiling of individual, line images.

It is appreciated, therefore, that in conventional sequential 1D hyper-spectral imaging the overall image acquisition process can take long time, for example at least several seconds. At the same time, typical latency and duration of saccades, that serve as a mechanism for fixation and rapid eye movements and cannot be controlled, are about 100 msec or even shorter and depend of frequency of the eye movement. At approximately equal time scale, lighting conditions may also change, unpredictably complicating the processing of acquired image data. As a result of the sequential nature of the hyper-spectral image collection, therefore, the eye must be immobilized for the duration of the imaging scan, which at least impedes the imaging procedure and often requires implementation of special means to prevent the cornea from drying. If the eye of the subject remains free to move, the resulting full image, taken line-by-line, is fractured in a fashion similar to that of a photograph that has been shredded into “stripes” and then has been reassembled or reconstructed without a frame of reference that is common to each stripe. It is clear, therefore, that conventionally-implemented multi-spectral imaging requiring reconstruction of the final image from the individually obtained spectral images into what is sometimes referred to as a “spectral cube” or a “composite image” (every portion of which contains the spectral information about the object) complicates the data acquisition procedure.

Embodiments of the present invention implement a seven-wavelength oxymetry methodology and facilitate the acquisition of OS-related information via imaging of the eye that is not impeded by the saccadic movements.

### Exemplary Embodiments of a Multi-Spectral Camera

Multi-spectral imaging (MSI) equips the analysis of specimens with computerized imaging systems by providing access to spectral distribution of an image at a pixel level. While there exists a variety of multispectral imaging systems, an operational aspect that is common to all of these systems is the capability to form a multispectral image. A multispectral image is one that captures image data at specific wavelengths or at specific spectral bandwidths across the electromagnetic spectrum. These wavelengths may be singled out by optical filters or by the use of other instruments capable of selecting a pre-determined spectral component including electromagnetic radiation at wavelengths beyond the range of visible light range, such as, for example, infrared (IR).

Embodiment of an imaging camera that may be used with the present invention stems from the realization that the use of a two-dimensional array of secondary objective lenses positioned so as to spatially split or segment the incoming beam substantially at the plane where an image of the entrance pupil of the primary objective of the imaging system is located significantly simplifies the multispectral multi-channel imaging system. In such a configuration, the array of secondary objectives performs the role of a beam-splitting means and there is no need in a separate beam-splitting component. As a result, folding of the optical path can be avoided. Additional advantages of this configuration include simplicity of assembly, modularity, and reconfigurability of the imaging system.

It is also realized that due to the very nature of conventional multi-channel systems, that are configured to maximize spatial resolution of the resulting images, axially-asymmetric spatial truncation of the incoming light distribution in such conventional systems should be avoided at all costs. Related art recognizes this limitation and admonishes specific configurations that spatially segment or split the incoming beam asymmetrically with respect to the optical axis of the system). In particular, related art refers to difficulties of proper correlation and registration of the images produced by imaging sub-portions of the so segmented incoming beam. In conventional multi-channel systems, precision and symmetry of positioning of beam-splitting components in a transverse (with respect to the optical axis of the system) plane substantially defines the resulting spatial resolution in the image plane. However, in applications that do not require imaging systems with maximized spatial resolution or that can employ imaging systems having spatial resolution below a pre-defined threshold, axially-asymmetric positioning of the secondary objectives forming multiple images in the imaging plane can be sufficient. Embodiments of the present invention and applications of these embodiments take advantage of such configuration.

FIG. 1, for example, illustrates, in side view, an embodiment 300 that includes a primary objective 302 producing, in light L₃₀₈, an intermediate image 304 of the object 308. As shown, the image 304 is formed in a back focal plane of the objective 302. An optical relay lens 310 is positioned so as to have the intermediate image 304 to be located in a front focal plane of the lens 310 and collimates light L₃₀₄ incoming from the intermediate image 304 into a beam 314. The collimated beam 314 is further intercepted by a multi-channel re-imaging sub-system 320 that incorporates a two-dimensional array 324 of optical spectral filters 328 and a two-dimensional array 330 of secondary objectives 334. Generally, the spectral filters 328 are optically different from one another so that each of them transmits light in a different portion of the optical spectrum. For example, the spectral filters 328 may be band-pass filters. Some of the filters, however, may have overlapping or even similar optical characteristics. According to the invention, the secondary objectives 334 spatially segment the beam 314 upon its traversal of the filter array 324 into a multitude of beam portions and each of the objectives 334 images a corresponding beam portion onto a detector element 340 that simultaneously records the corresponding images 344. Each of the individual images 344, therefore, is formed in a portion of the optical spectrum that corresponds to the spectral transmission characteristic of an associated filter 328.

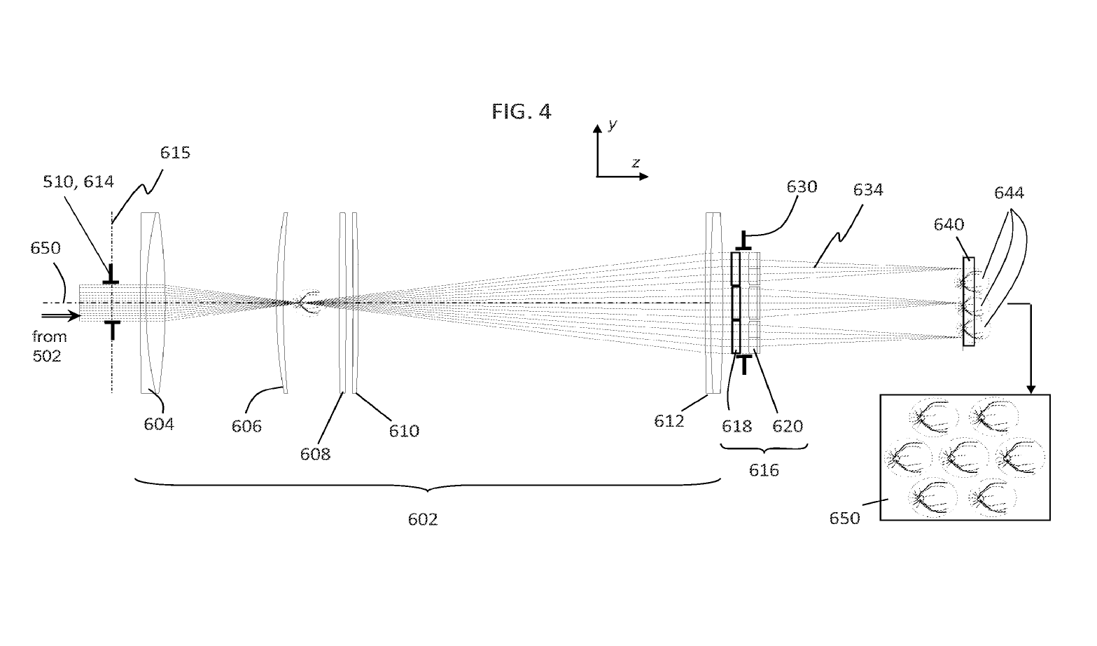

The detector element 340 may be a single detector such as a CCD disposed in a back focal plane of the array 330 that contains substantially optically identical objectives 334. In reference to FIG. 4, an image frame 350 containing individual spectral images 344 is further developed, 452, with a computer processor and the use of appropriate data-processing algorithms to produce a resulting aggregate multispectral image frame 454. In another embodiment (not shown), the array 330 may contain the secondary objectives possessing different optical characteristics (such as different focal lengths and/or clear apertures, for example) and forming corresponding images 344 in a plurality of differing focal planes corresponding to different objectives 334. In this configuration, different spectral image frames are recorded using a single detector or a plurality of detectors and further independently processed to form the resulting multispectral image. In a specific embodiment, the secondary objectives 334 of the re-imaging subsystem 320 may have adjustable focal lengths to compensate for manufacturing tolerances and other variations of systemic parameters and filters 328 have pass-bandwidths that do not overlap. While the embodiment 300 illustrates a case where the number of either filters 328 or objectives 334 is three, the maximum number of filters or secondary objective in the sub-system is determined by the imaging application and the minimum number is two.

In further reference to FIG. 1, the re-imaging sub-system 320 may be configured so as to have entrance pupils of the objectives 334 aligned in the same plane 354 that is axially disposed, within the embodiment, to coincide with an exit pupil 360. The exit pupil 360 is an image of an entrance pupil 362 (which may be an aperture stop) formed by a telecentrically operating sub-system 364 formed by a combination of the primary objective 302 and the relay lens 310. The position and size of the exit pupil 360 is calculated as known in the art based on optical characteristics of the relay sub-system 364. The parameters of the relay sub-system 364 are preferably chosen so as to assure that the exit pupil 360 is bigger than the entrance pupil 362 in order not to restrict the transverse dimensions of the re-imaging sub-system 320. The optical characteristics of the array 330 are preferably selected to produce the images 344 with dimensions and spacings among them that are optimized for the chosen pixel count of the focal-plane detector array 340. Additional design considerations include the spectral range of the detector array 340.

Generally, the optical components of embodiments of the present invention may be made of optical quality glass, crystals, or polymeric materials, or of any other materials possessing optical quality in transmission of light. The focal-plane 2D-array 340 can be CCD, CMOS, InGaAs, InSb, or any other type of focal plane arrays used for purposes of detecting light.

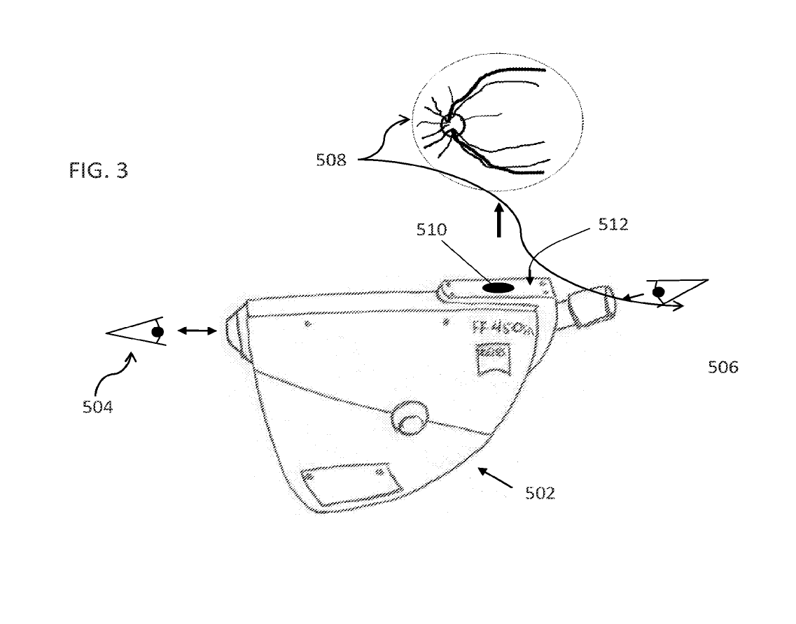

An alternative embodiment of the present invention is further described in reference to FIGS. 3 through 6. This embodiment is used to generate a multi-spectral image of the human retina using seven discreet spectral bands defined between 520 nm and 590 nm that are judiciously selected to measure the oxygen saturation of the hemoglobin in retinal vessels and tissues. It is appreciated, however, that embodiments described below may be appropriately modified to define a different number of spectral channels (for example, 3, 5, 10 or another number).

In general, embodiments of the invention can be adapted to operate in conjunction with commercially-available imaging devices such as, e.g., a fundus imaging device 502 (Zeiss FF450 IR) schematically shown in FIG. 1 conventionally used to capture standard images of the human retina, for clinical purposes, such as fluorescence angiograms (either polychromatic or monochromatic), fundus photographs, red free images, autofluorescent images, or indocyanine green angiograpms, for example. FIG. 3 schematically illustrates the standard fundus imaging instrument 502. Traditionally, the standard imaging device 502 is used as follows. A patient having an eye 504 sits in front of the instrument 502 while a medical personnel 506 aligns the device so as to capture an image of the retina 508 of the eye 504 through the exit pupil 510 of the device 502 with a focal-plane detector (not shown). The top port 512 of the fundus imaging instrument 502 where the exit pupil 510 is located is usually configured to accommodate different types of two- dimensional focal-plane arrays, such as the array 340 of FIG. 1, depending on the type of clinical information that has to be obtained from the resulting image.

In a particular application contemplated by the present invention, and in reference to FIGS. 3 and 4, an alternative embodiment of the invention is disposed in place of a detector array of the fundus imaging device 502, in proximity to the exit pupil 510 of the device 502. As shown in FIG. 4 and in comparison with the embodiment 300 of FIG. 1, the relay sub-system 602 includes five different lens elements 604, 606, 608, 620, and 612 and has an entrance pupil 614. The relay sub-system 602 is disposed, with respect to the relay subsystem 602, so as to have the exit pupil 510 of the instrument 502 (not shown) and the entrance pupil 614 of the relay sub-system 602 coincide in the same plane 615. A re-imaging sub-system 616, which includes an bandpass filter array 618 and an array 620 of secondary objectives, is positioned at an exit pupil 630 of the relay sub-system 602, as described in reference to FIG. 1. Each of the individual objectives of the array 620 focus the light 634 on a 2D-focal plane detector array 640 to produce individual spectral images 644 of retina of the eye 504. The individual images 644 are further shown in an image frame 650. A more detailed perspective view of the re-imaging subsystem 616 is shown in FIG. 5.

In reference to FIG. 5, the five elements 604, 606, 608, 610, and 612 of the relay sub-system 602 can be divided into three groups: a first (positive) group with the primary objective 604, a second (correction) group with the lenses 606, 608, and 610, and a third (positive) group with the lens 612. The first and third groups operate to re-image and expand the exit pupil 510 of the fundus imaging device 502 into the exit pupil 630 of the relay sub-system 602. The second, correction group is configured to correct optical aberrations that would otherwise render the optical quality of the images 644 unusable. The transverse (with respect to an optical axis 650) dimensions of the exit pupil 630 are preferably slightly larger than those of the re-imaging sub-system 616 to avoid vignetting and/or shadowing effects.

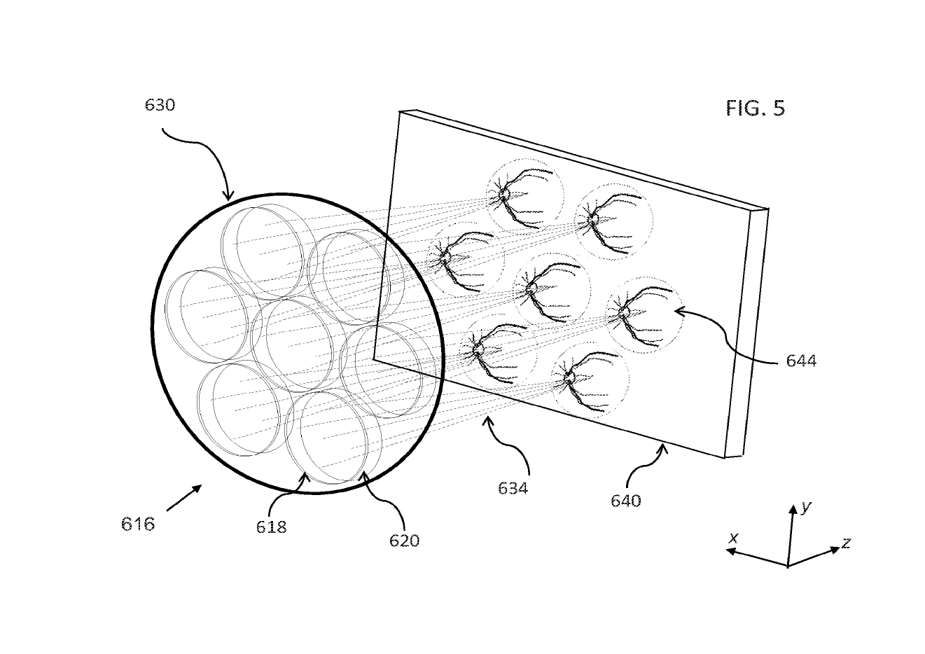

FIG. 6 schematically illustrates that the image frame 640, containing a multitude of individual images 644 each of which retains individual spectral responses of corresponding individual bandpass filters 618, is further split into individual images that are processed 802 and recombined, with the use of computer processor, into a final composite multi-spectral image 804.

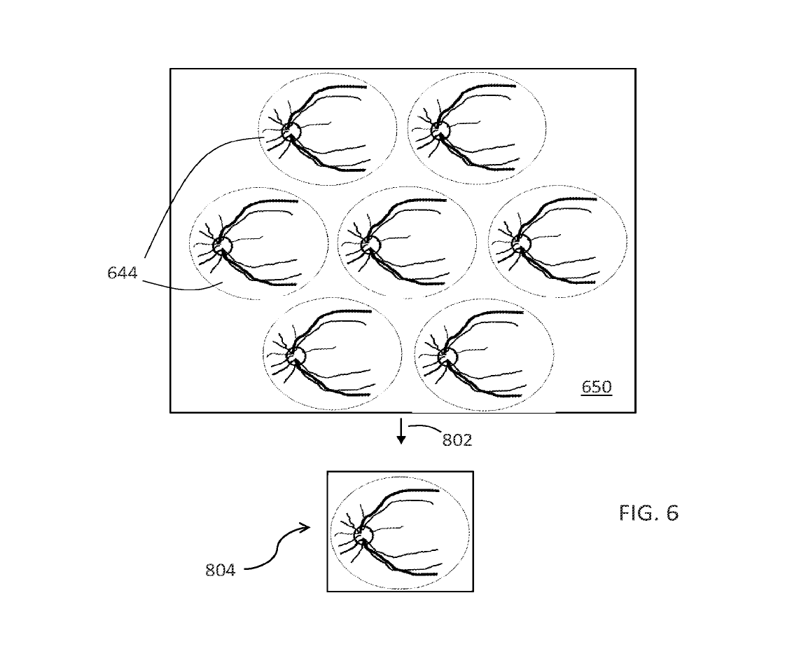

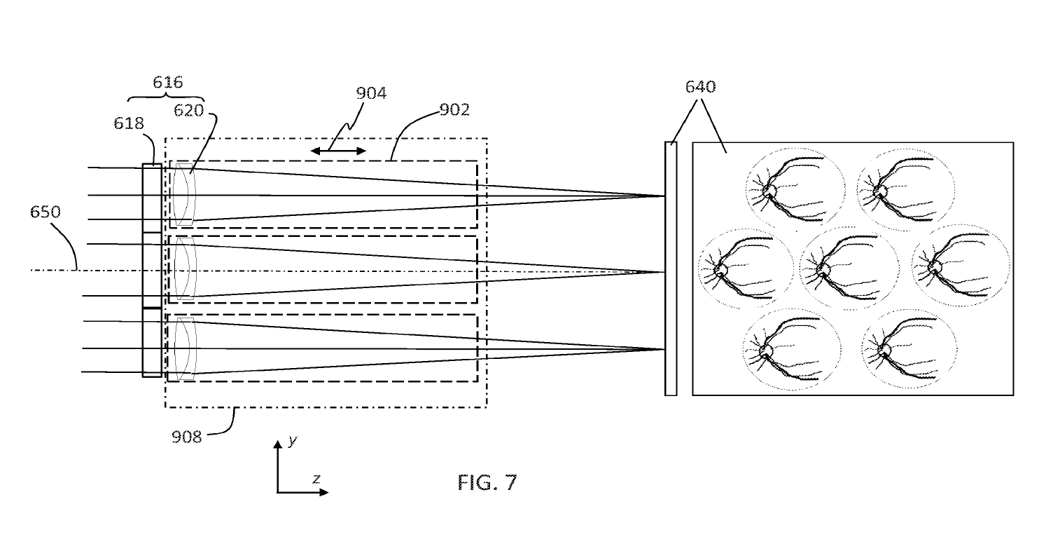

As shown in FIG. 7, each of the individual secondary objective lenses 620 of the re-imaging sub-system 616 is individually repositionable via an associated elongated lens barrel 902 that provides a means for re-adjusting a position of the given secondary objective lens 620 along the optical axis 650 in order to obtain a highly focused corresponding individual 644 (not shown in FIG. 7) on the image frame 640. Such repositioning is illustrated with an arrow 904. A plurality of the lens barrels 902 is assembled in a single mount 908 that utilizes a barrel-locking mechanism in order to maintain the position of the lens barrels after the focus adjustment.

As discussed herein, the fact that spatial segmentation of the incoming beam is achieved by using secondary objectives, of the re-imaging sub-system, as a spatial beam-splitting means at the location of the exit pupil of the relay-subsystem of the present invention allows to avoid the use of auxiliary beam-splitting components used in prior art and, therefore, increases the system tolerance to mechanical misalignments.

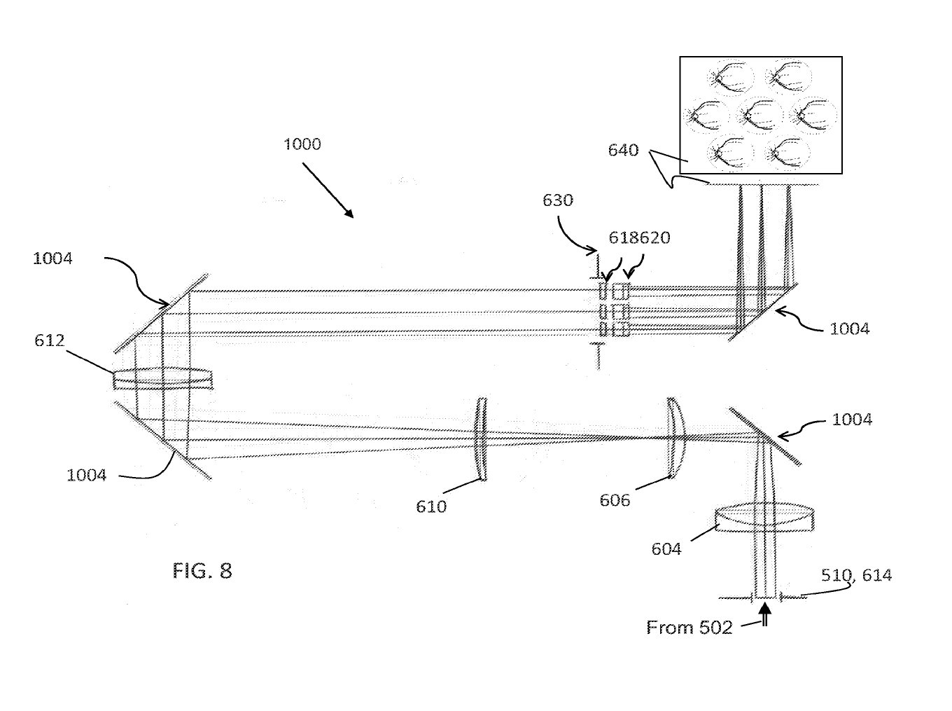

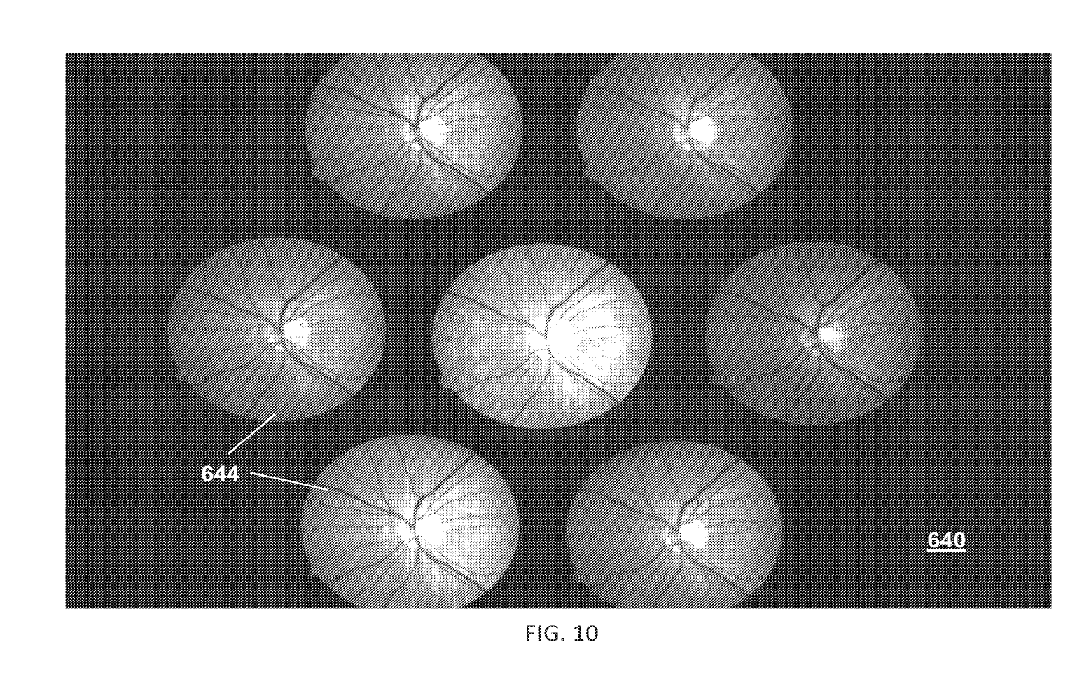

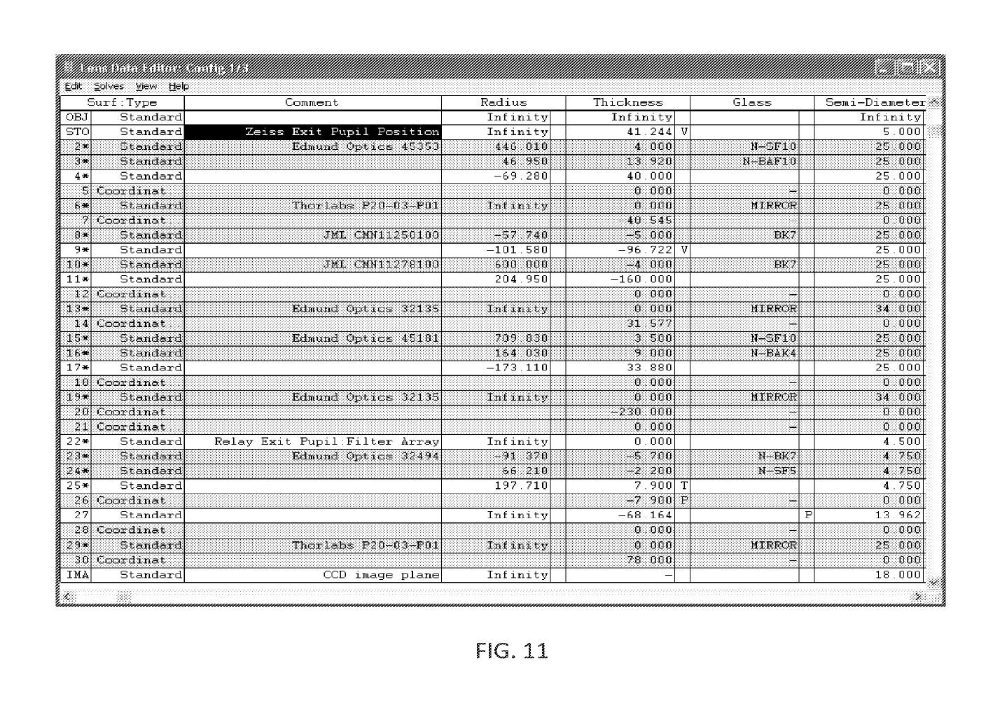

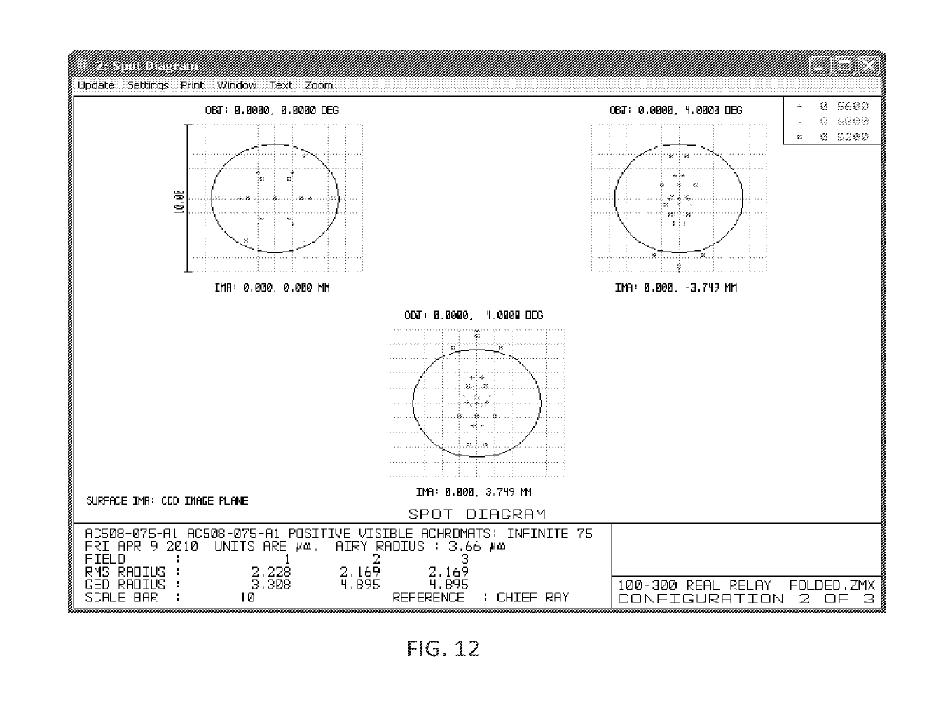

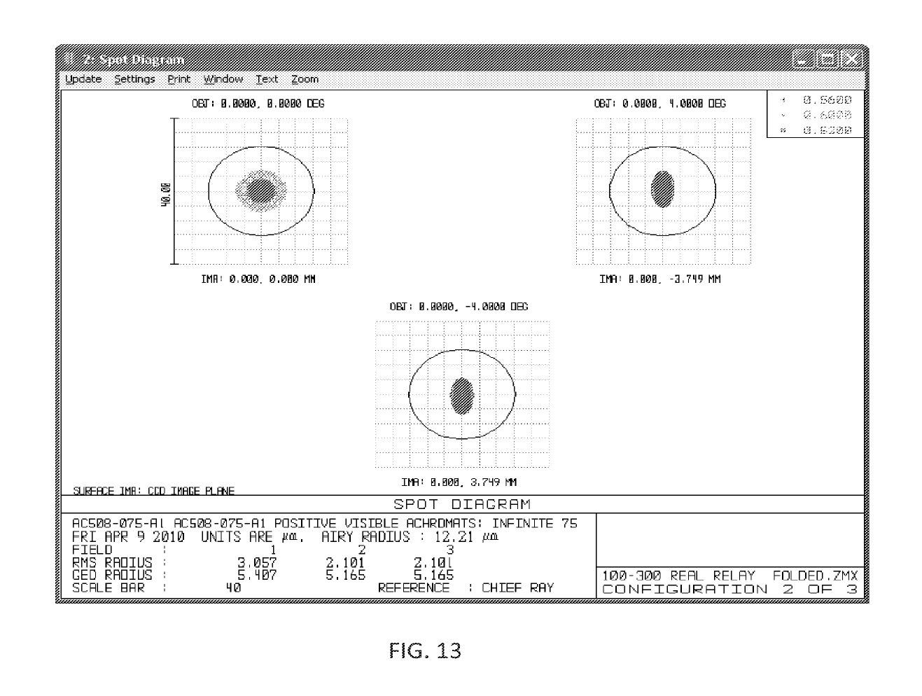

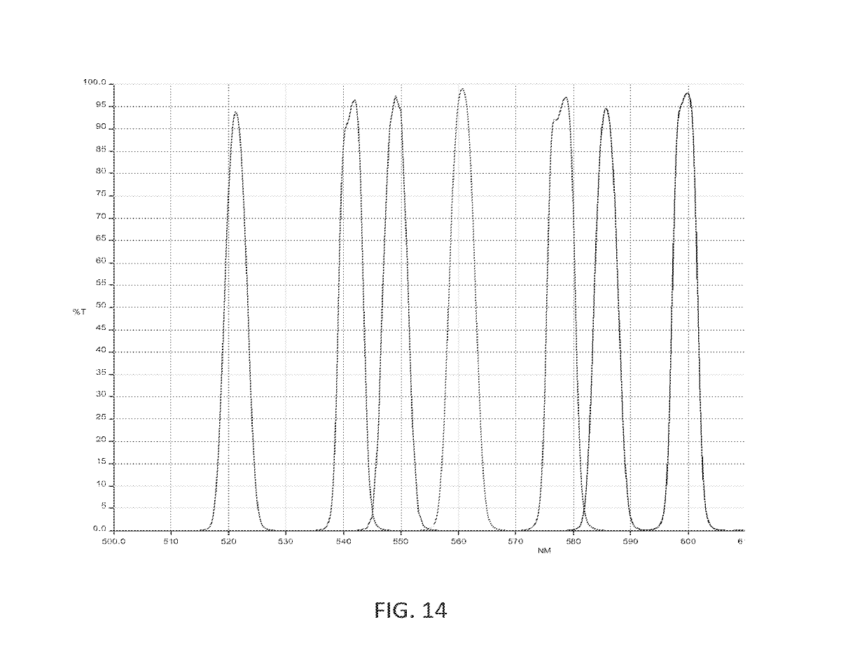

A specific embodiment 1000 of the camera is shown in FIG. 8 to be similar to the embodiment 600 of FIG. 4. This embodiment is specifically adapted to assure that the optical path of the overall system is folded to make the system more compact. This, in practice, facilitates integration of the embodiment to an existing imaging device such as the fundus imaging device 502. According to the embodiment 1000 and in comparison with the embodiment 600, several adjustable folding mirrors 1004 are utilized and one lens of the relay sub-system 602 (lens 608) is removed to accommodate the preferred optical layout. The array 618 contains seven optical filters having corresponding bandwidths of about 4 nm centered at 522 nm, 542 nm, 548 nm, 560 nm, 569 nm, 577 nm, and 586 nm, respectively. Optical design parameters for this embodiment are summarized in FIGS. 11 through 14. FIG. 11 illustrates the geometrical layout of the embodiment, while FIG. 14 presents transmission characteristics of the optical filters of the array 618. FIGS. 12 and 13 illustrate spot diagrams associated with an imaging system having a full aperture and that of an embodiment of the present invention with a segmented aperture, respectively. As expected, the diffraction limit of the resulting image of the segmented aperture system is, as expected, slightly lower than that of a full-aperture system where the exit pupil of the relay sub-system is not spatially segmented (12.21 microns and 3.66 microns, respectively). As discussed above, however, this reduction in spatial resolution of the embodiment of the invention does not affect the use of the embodiment in ophthalmologic imaging discussed below, where the spatial resolution of the eye remains a limiting factor.

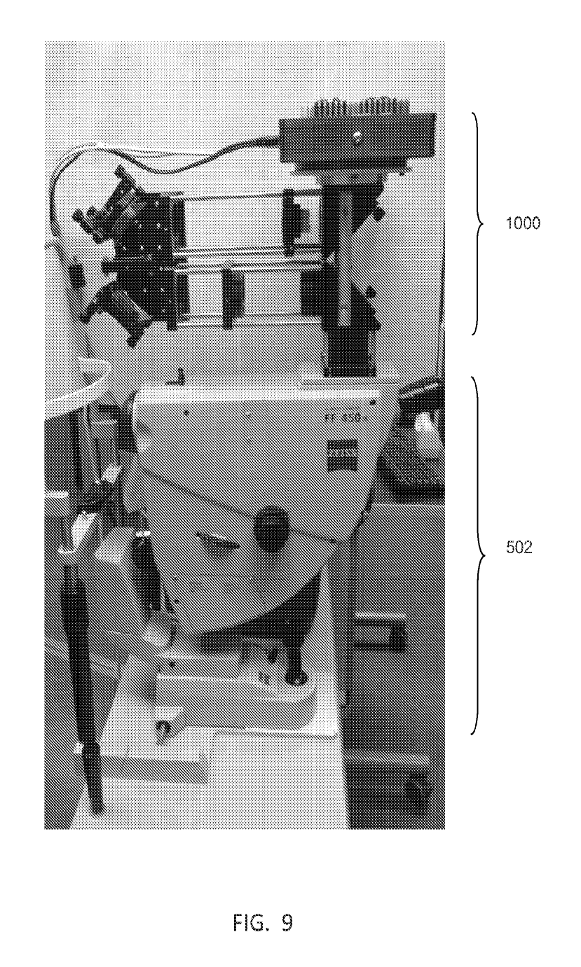

FIGS. 9 and FIG. 10 illustrate an example of practical integration of the embodiment 1000 with the fundus imaging device 502 and the resulting image frame 650 with individually registered spectrally-separate images 644.

The advantages of embodiments of an imaging system described above include, without limitation, a recordation of a complete spectral image with the use of a two-dimensional focal-plane detector array in a single exposure, without the need in spatially deviating the image-forming beams from one another. Advantages of using embodiment of the present invention are particularly pronounced in applications that involve imaging of dynamic objects, for example moving objects. This greatly helps in the recombination of the individual images into a single spectral image.

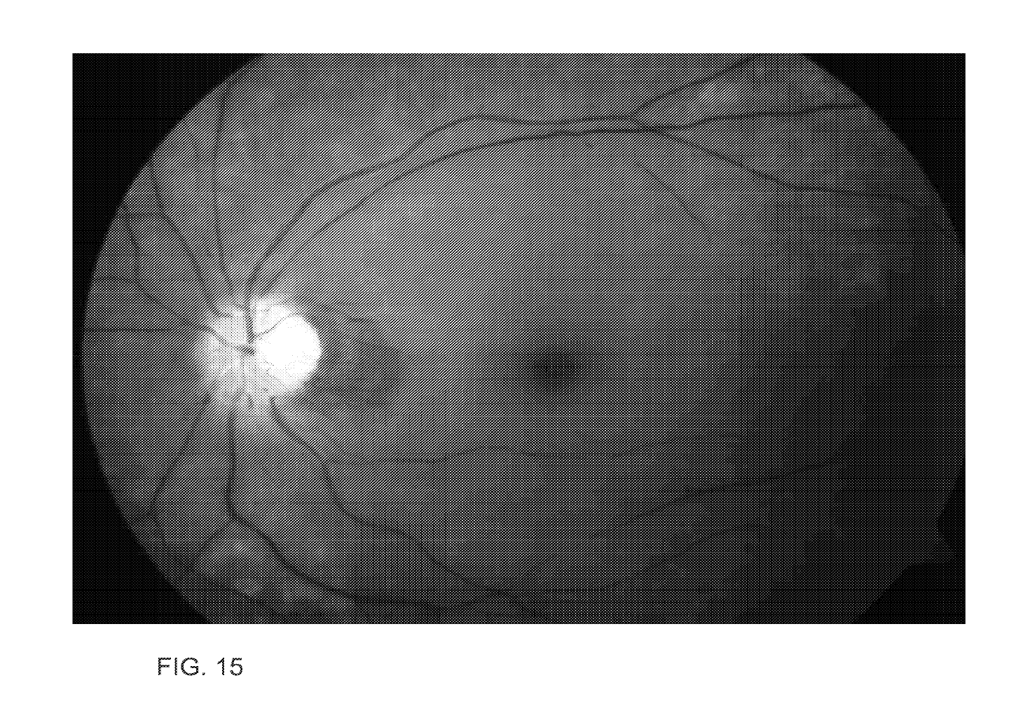

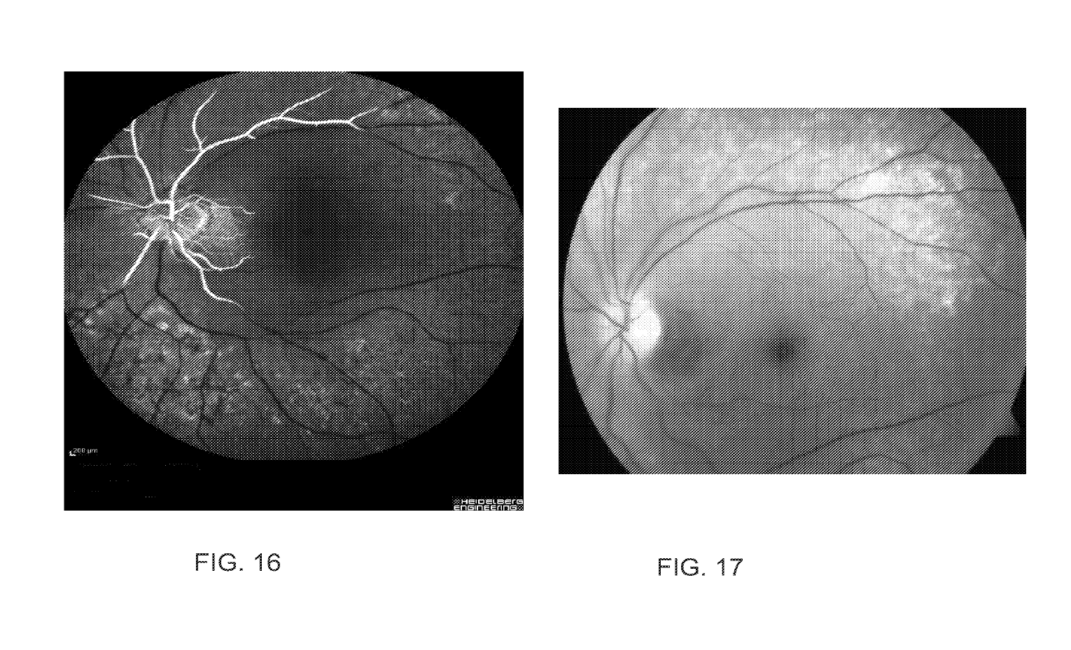

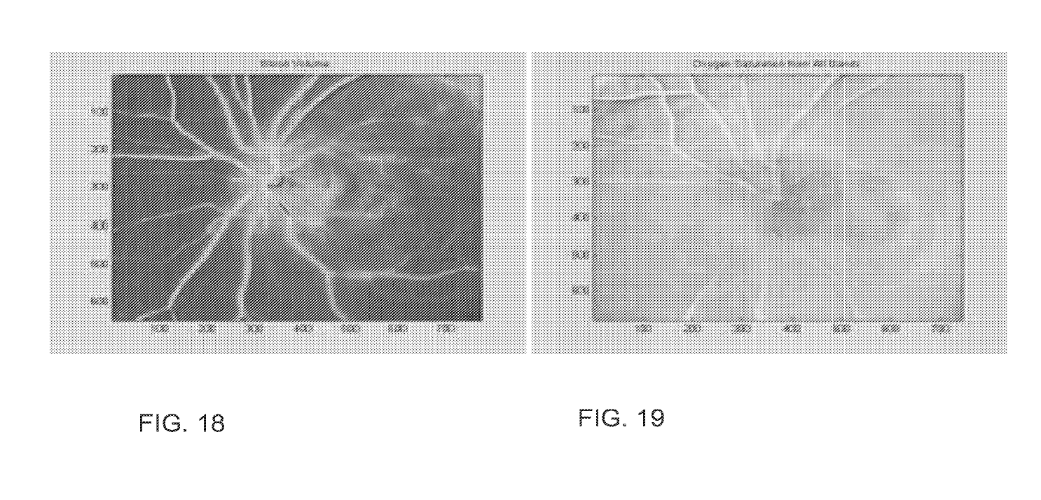

FIGS. 15 through 19 provide examples of retinal images, obtained with an embodiment of a camera of the invention, of a patient with Central Retinal Artery Occlusion (CRAO). CRAO can occur due to an embolus or an obstruction caused by inflammatory or other pathological processes such as tumors. FIG. 15 displays a fundus color photograph of the left eye of a patient with acute CRAO, demonstrating whitening and edema of retinal tissue and presence of red spot in the center (“cherry red spot”) due to arterial obstruction. FIG. 16 is a fluorescent angiography image that confirms the arterial obstruction. As shown, the macular area (central part) is dark thus indicating the lack of blood and oxygenation. FIG. 17 is an 8-day fundus photograph, that shows the same eye with more visible arterial circulation and, therefore, better blood supply and oxygenation. FIG. 18 corresponds to images of FIGS. 15 and 16 and shows an analyzed aggregate multi-spectral image obtained with an embodiment of the invention. This image demonstrates relative oxygen saturation of retinal blood vessels and tissues. The blue coloration of most of the image corresponds to areas of oxygen desaturation and, therefore, low oxygen saturation or hypoxia. Yellow or red coloration areas in the very central portion of the image indicate higher oxygen saturation and confirms the information obtained with image of FIG. 16, where fluorescent angiography shows some potency of the blood vessels at the optic nerve level. This confirms clinical findings of fundus photography and fluorescent angiography finding. An image of FIG. 19 is the analyzed aggregate multi-spectral image of the same patient in obtained in a follow-up testing, after the retinal circulation has improved. The areas of yellow and red show higher oxygen saturation. This confirms the clinical findings obtained from image of FIG. 17.

### Exemplary Embodiments.

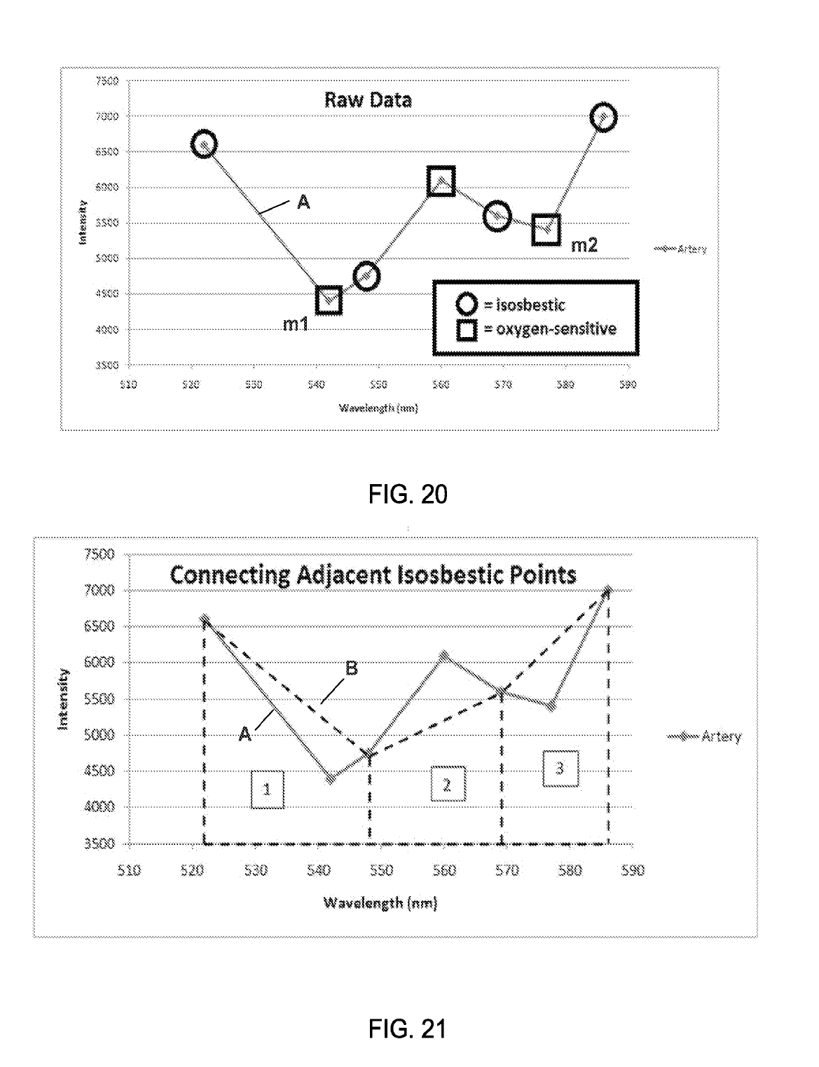

While the discussion below mainly refers to processing of data acquired through imaging of retina-related biological tissue, it is done only for the sake of simplicity of the discussion, and the processing of data related to images of an ocular tissue or a component of an eye in general is within the scope of the invention. According to an embodiment of the invention, optical data characterizing retinal scene in different spectral bands are acquired simultaneously (for example, with an embodiment of the imaging system discussed above or a similarly performing imaging system). Generally, optical data is acquired with a use of an optical system including a beam-splitting arrangement that divides in incoming light wavefront into a plurality of optical beams each of which defines a specific spectral bandwidth. In one, such acquisition may occur within a time period that is shorter than a duration of a typical saccade, and, optionally, at several discrete wavelengths. In a specific embodiment, the time-length of a snap-shot imaging is no more than about 10 msec, and preferably less than 5 msec. The acquired data are then processed, as presented below, to determine the OS level of the retinal scene. Such method of data acquisition facilitates performing a retinal diagnostic on the subject without immobilizing the subject's eye. For example, a single snapshot of the retina taken by an imaging camera such as that described above provides, at an output, seven discrete 2D images at seven pre-determined wavelengths (referred to as isosbestic wavelengths of 522 nm, 548 nm, 569 nm, and 586 nm; and oxygen-sensitive wavelengths of 54 nm, 560 nm, and 577 nm) that define a discrete approximation of the reflectance spectrum of the retina. The image data contained in such a single snapshot also allows to determine the relative values of intensity of light reflected by the imaged tissue (such as an ONH) at the pre-determined wavelengths, which are then plotted in arbitrary units of intensity as a function of wavelength, as shown by curve A in FIG. 20. As shown, curve A illustrates a discrete reflectance spectrum of saturated (HbO₂ signature) retina that is defined by the above-mentioned pre-determined wavelengths and sufficiently well approximates, for intended purpose, a corresponding continuous reflectance spectrum and that defines two minima, m1 and m2, respectively corresponding to (oxygen-sensitive) wavelengths of peaks of light absorption by such saturated retinal blood. (A discrete reflectance spectrum of unsaturated (Hb signature) blood can be obtained in a similar fashion.)

As used for the purposes of the description and the appended claims, an isosbestic wavelength is a wavelength at which reflectance spectra of oxygen saturated (HbO₂ signature) and oxygen-unsaturated (Hb signature) blood in biological tissue, measured under otherwise equal conditions, have equal values. The HbO₂-signature curve typically contains two minima respectively corresponding to wavelengths of peaks of light absorption by the saturated retinal blood, while the Hb-signature curve typically has a single broad minimum. A point on a reflectance spectrum of a given blood sample that corresponds to an isosbestic wavelength is referred to as an isosbestic point.

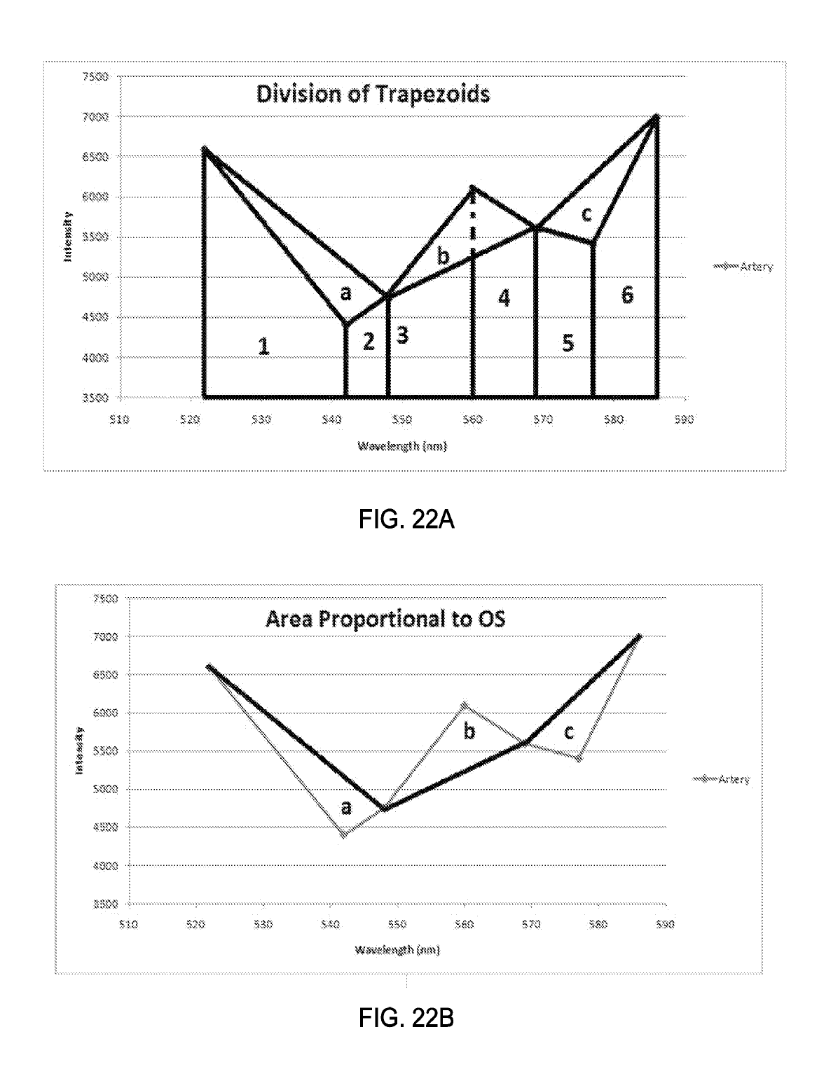

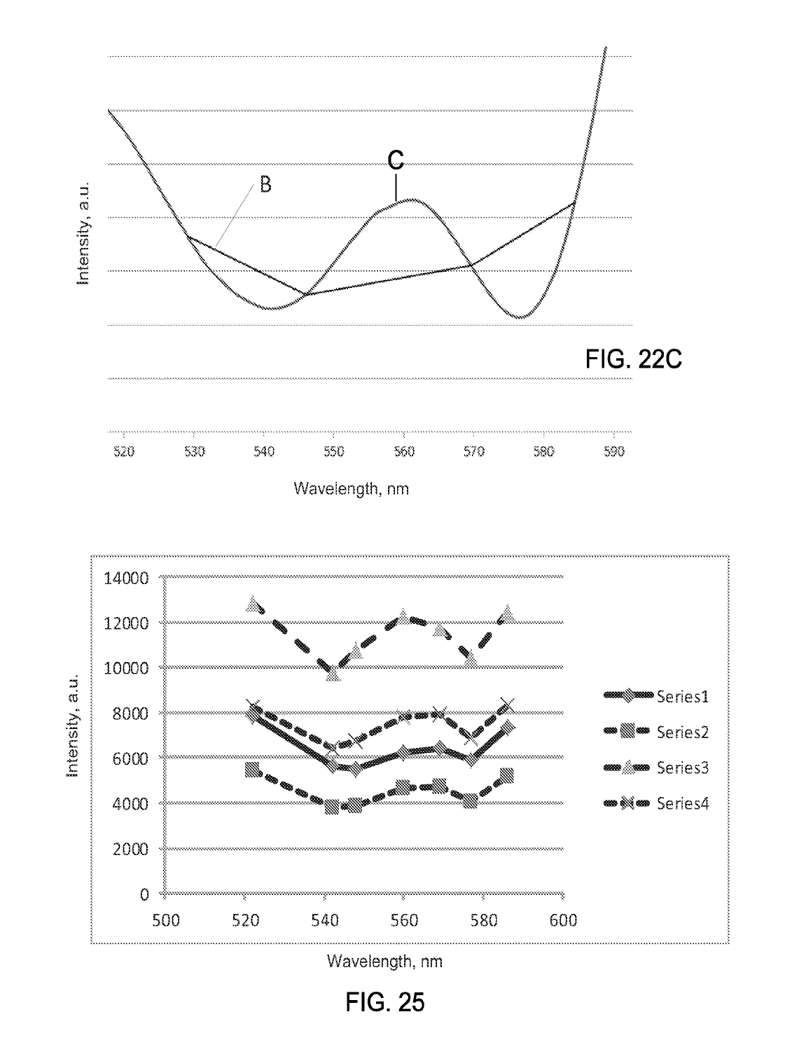

Sequentially connecting the isosbestic points of curve A further defines curve B (shown as a dotted line in FIG. 21 and referred to hereinafter as an isosbestic line) that, in conjunction with curve A, is used to determine the overall area between the curves A and B that is found to be proportional to the relative OS value of the imaged scene. The graphical determination of the sought relative OS value is schematically illustrated in FIGS. 22A and 22B, and includes appropriate subtraction and addition of areas of several trapezoids (shown as 1, 2, 3) to obtain the areas of triangles a, b, and c, as shown in the example of FIG. 22B. In order to determine the OS value that is normalized with respect to the optical signal used for acquiring the reflectance spectrum, one should determine the absolute value of the of the area bound by the curves A and B. To this end, the triangular areas defined below the isosbestic line B (areas a, c) are accounted for as areas having a different sign as compared to that of the area defined above the isosbestic line B (area b).

To appreciate the significance of defining the discrete reflectance spectrum via measurements of light intensity reflected off the retina at seven pre-determined wavelengths, in accordance with an embodiment of the present invention, FIG. 22C illustrates a continuous reflectance spectrum of saturated (HbO₂ signature) retina, shown as a W-shaped curve C. From the comparison of the FIGS. 21, 22A, 22B, and 22C one can recognize that, in order to optimize the accuracy and precision of determination of the OS value based on the discrete reflectance spectrum A and the isosbestic line B, all three areas a, b, c should be taken into account in the calculation. Defining all three areas a,b,c requires, in turn, a determination of at least seven discrete wavelengths, as discussed above. While it is possible to use a different number of wavelengths to establish a discrete reflectance spectrum, if such number is smaller than seven (for example, five), the resulting five-point discrete spectrum would not follow, even approximately, the W-shape of a continuous spectrum C. Therefore, the resulting five-point spectrum and the isosbestic line would not enclose the areas defined by undulation of the W-shaped continuous spectrum, and will define not three but only two areas between them. Consequently, the error in derivation of the OS value will increase. If the number of pre-determined wavelengths is reduced even further, the calculation error may render the determined OS values to be impractical, for intended purpose. As an example, an error in determination of the OS value with the use of a seven-point discrete reflectance spectrum in accordance with an embodiment of the present invention is empirically shown to be about 1%. In contradistinction, if only three wavelengths were used to define a discrete reflectance spectrum of the retina, there would be only one—not three—distinct areas defined by the isosbestic line and such discrete reflectance spectrum and, accordingly, the error in calculation of the OS value would grow to at least 7%. On the other hand, the use of a greater number of discrete wavelengths (for example, 10 or 14 or any other number) at which the discrete reflectance spectrum of the retina is measured, while still within the scope of the invention, increases the measurement (acquisition and registration) error due to increase in time that is required to experimentally acquire the measurement data and the increased difficulty due to the need to register a larger number of images. In addition, since it may be preferred to acquire all the images within a period of time less than a duration of a saccade, as the number of wavelengths at which the images are to be acquired increases, the acquisition time approaches the duration of the saccade, which makes the use of the larger number of discrete acquisition wavelengths less advantageous.

A further data normalization step may be required in order to be able to compare the relative OS values determined with the use of different blood volumes. For this purpose, the relative perfusion index (RPI), defined as

RPI=7/2*(I₅₂₂+I₅₈₆)/(I₅₂₂+I₅₄₂+I₅₄₈+I₅₆₀+I₅₆₉+I₅₇₇+I₅₈₆),

where Iᵢⱼₖ is a detected intensity at a wavelength of ijk nm, is used as a normalizing coefficient. The relative OS value of the imaged scene (such as an ONH) that is normalized with respect to the volume of blood in the imaged scene is obtained by dividing the previously determined normalized areas a, b, and c by the RPI.

Percent OS value is calculated from groups of pixels associated with separately imaged tissue components using any appropriately fitted (with the use of, e.g., a linear least square curve fit model) recorded hemoglobin spectrum to reference spectral curves acquired with the use of fully oxygenated (substantially 100% oxygenation level) and deoxygenated (substantially 0% oxygenation level) blood. An assumption that the arterial blood has an OS of 98% was used.

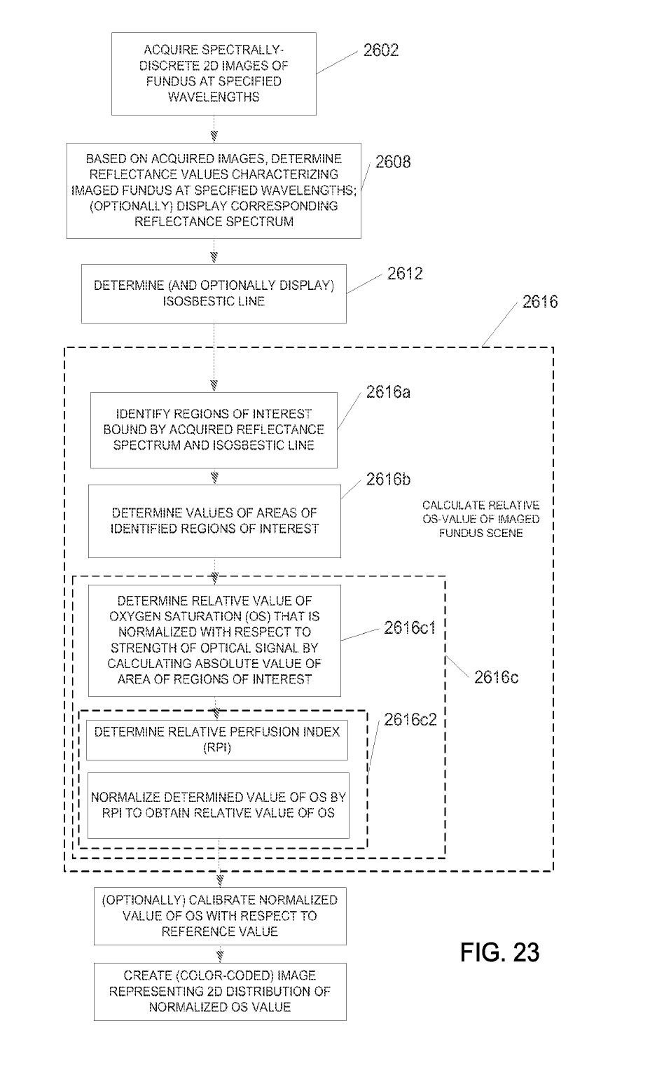

FIG. 23 presents a schematic flow-chart corresponding to the process for determining a relative value of oxygenation (the OS value) of blood in a scene that includes a portion of the visual system (fundus, such as retina or ONH) imaged with the use of an embodiment of the invention. It is appreciated that an embodiment of the algorithm was implemented with a computer processor specifically programmed to perform at least the following steps. Following the acquisition 2602, at predetermined wavelengths, of spectrally discrete images of the scene (such as fundus, e.g. ONH or retina) with an appropriately chosen detector, the values of reflectance of the scene at those wavelengths are determined, on a pixel-by-pixel basis or as averaged across the area of the detector values, and optionally displayed as a function of wavelength. As a result, at step 2608, a relative discrete reflectance spectrum of the imaged scene is formed (and referred to herein, for simplicity, as an acquired reflectance spectrum). In an alternative embodiment, not shown, the acquisition and display of the acquired reflectance spectrum of the imaged fundus can be performed contemporaneously with acquisition of the spectrally-discrete images.) Determination of an isosbestic line is further carried out at step 2612 by sequentially connecting those spectral points of the formed acquired reflectance spectrum that correspond to pre-determined isosbestic wavelengths. The following, at step 2616, calculation of relative value of OS may include determining, at sub-step 2616a, the regions bound by the acquired reflectance spectrum and the isosbestic line and determining the corresponding areas of these regions, 2616b. The calculation may further include an optional normalization sub-step 2616c including a determination of the value that represents relative OS and that is normalized with respect to both the strength of the optical signal, used to acquire the reflectance spectrum, and the amount of blood contained within the imaged scene. Such normalization is accomplished by: (i) determining, 2616c1, the absolute value of an aggregate area representing the regions bound by the acquired reflectance spectrum and the isosbestic curve (which includes taking into account the arithmetic signs of the values that are defined, for a given region, by whether a corresponding region-defining section of the isosbestic line lies above or below a corresponding region-defining section of the acquired reflectance spectrum); and (ii) normalizing, 2616c2, the determined absolute value of an aggregate area by a relative perfusion index determined from the acquired reflectance spectrum. At a final optional step, 2620, the normalized relative value of OS can be further scaled with respect to a judiciously chosen reference value. In one embodiment, e.g., this reference value may be chosen to represent the OS value of arterial blood (98%, for example). It is appreciated that the use of a bigger number of reference values (e.g., several values of OS for veins and/or any other blood vessels for which the percentage of OS is known) may allow to perform a reliable data fit of the experimentally obtained OS-related data. Finally, at step 2630, an image representing a normalized value of OS averaged across the area of the imaged tissue, or a 2D distribution of OS values across that area is optionally presented to and saved for the user in any form including a visual image on a display, a hard copy print, or an array of values stored on a tangible non-transitory computer-readable medium.

An embodiment of the invention has been described as including a processor controlled by instructions stored in a memory. The memory may be random access memory (RAM), read-only memory (ROM), flash memory or any other memory, or combination thereof, suitable for storing control software or other instructions and data. Some of the functions performed by embodiments have been described with reference to flowcharts and/or block diagrams. Those skilled in the art should readily appreciate that functions, operations, decisions, etc. of all or a portion of each block, or a combination of blocks, of the flowcharts or block diagrams may be implemented as computer program instructions, software, hardware, firmware or combinations thereof Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

### EXPERIMENTAL EXAMPLES

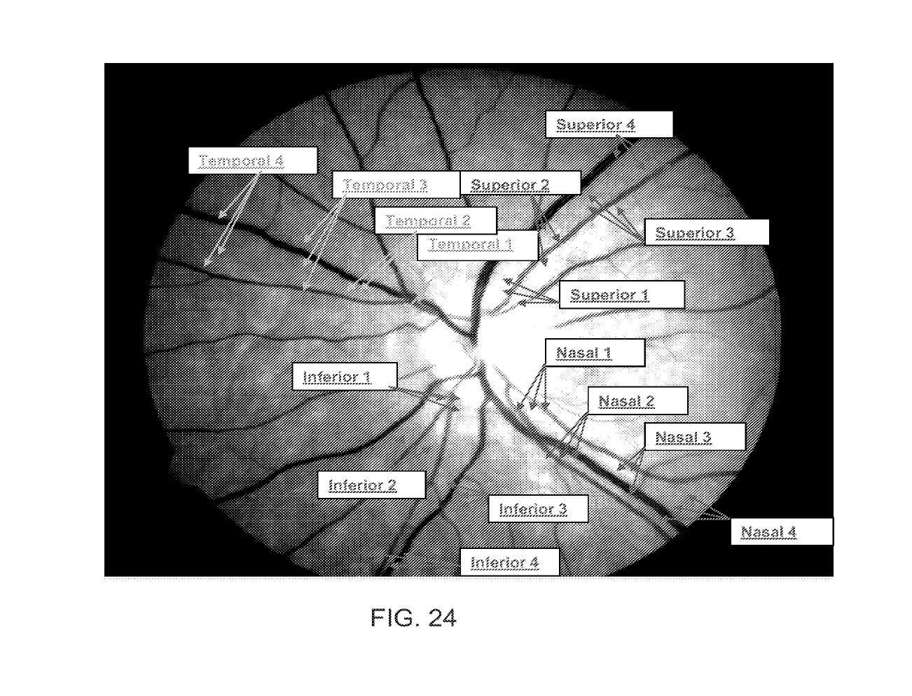

FIG. 24 and Tables 1 through 5 illustrate practical application of the method of the invention. FIG. 24 shows the areas of the ONH imaged with the use of an embodiment of the imaging camera described in reference to FIGS. 3 through 9, at seven pre-defined wavelengths identified above. The measurement areas are identified, across the image, by approximately concentric annuli not shown), with area 1 being the inner circular portion of the ONH, area 2 being the adjoining annulus with a bigger radius, area 4 being the annulus with an outermost radius, and area 3 lying between areas 2 and 4. Average values of OS of blood in imaged tissue was determined with an embodiment of the method of the invention discussed herein for veins and tissue regions corresponding to areas 1 through 4 are summarized in Tables 1 through 4, respectively, under the assumption that the arterial blood has an OS value of 98%. Table 5 summarizes the results by grouping the data according to vein regions (temporal, superior, inferior, and nasal) and shows that the averages OS value for blood in those veins was determined to be 60.53%, whereas the OS value of the tissue was 75.78%. The experimental verification, of embodiments of the multi-spectral imaging system and/or data processing algorithm discussed above, independently repeated several days apart convincingly demonstrates that embodiments of the invention can be used for measuring OS if biological tissue (open microcirculation) and, in particular, of the retina, the optic nerve head and overlying artery and vein.

FIG. 25 illustrates additional discrete reflectance spectra for blood in veins and arteries, obtained as a readout from 16-bit CCD camera, with the 16 bit being equivalent to the relative intensity value of 65536. Table 6 summarizes the readout intensity values.

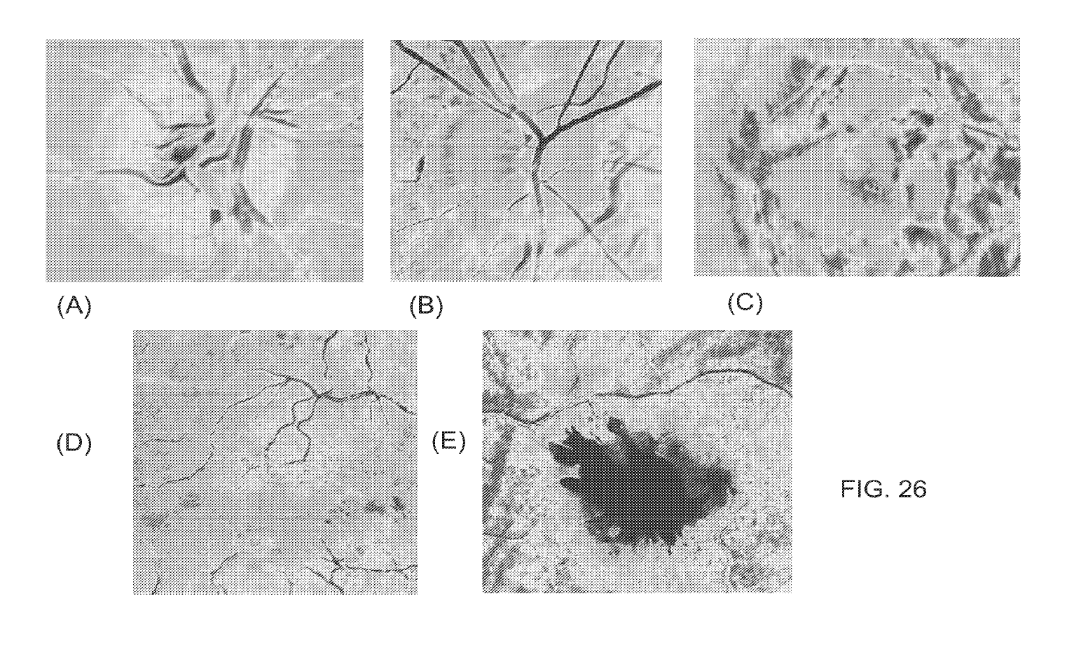

Distribution of the relative OS values across the imaged scene (e.g., an ONH) can be further used to produce 2D maps of oxygen saturation values across the region of interest of the retina. The maps may include arrays of data, or, alternatively, may include color-coded 2D images of the retinal ROI where the color-coding represents the levels of oxygen saturation. FIGS. 26A, 26B, 26C, 26D, 26E offer a visual comparison between such OS-maps created, with the use of embodiments of the method of the invention as applied to experimental data obtained with embodiment of the camera described herein, for ONH in two situations. It is appreciated that, in order to create the 2D distribution of OS values across the imaged ONH, the transformation of image data representing the acquired reflectance spectrum is carried out on a pixel-by-pixel basis. The scale of OS values in the examples of FIGS. 26A, 26B, 26C, 26D, 26E is established through color-coding chosen, in this example, such as to have the degree of red coloration illustrate the increase of OS and the blue coloration represent smaller OS levels. FIG. 26A shows a distribution of relative OS values characterizing an optic nerve head in a normal subject. In comparison, FIG. 26B shows a distribution of relative OS values from the optic nerve head in a subject with wet aged-macular degeneration (AMD). FIG. 26C shows a distribution of relative OS values characterizing the macular region (center part of retina) in a normal subject. In comparison, FIGS. 26D and 26E show distributions of relative OS values from the macular region in a subject with dry and wet aged-macular degeneration, respectively.

TABLE 1
Area 1 Summary

| | Day 1 | Day 7 | Average ± SD of | Day 1 | Day 7 | Average ± SD of |
|---|---|---|---|---|---|---|
| Data Set | Vein | Vein | Mean | Tissue | Tissue | Mean |
| Temporal | 68.89% | 67.69% | 68.29% ± 0.85% | 78.04% | 82.33% | 80.19% ± 3.03% |
| Inferior | 68.75% | 60.29% | 64.52% ± 5.98% | 79.95% | 72.28% | 76.12% ± 5.42% |
| Nasal | 68.40% | 62.71% | 65.56% ± 4.02% | 77.73% | 74.05% | 78.89% ± 4.98% |
| Superior | 59.66% | 54.02% | 56.84% ± 3.99% | 77.70% | 71.22% | 74.46% ± 4.58% |

TABLE 2
Area 2 Summary

| | Day 1 | Day 7 | Average ± SD of | Day 1 | Day 7 | Average ± SD of |
|---|---|---|---|---|---|---|
| Data Set | Vein | Vein | Mean | Tissue | Tissue | Mean |
| Temporal | 64.51% | 64.85% | 64.68% ± 0.24% | 74.35% | 75.94% | 75.15% ± 1.12% |
| Inferior | 64.70% | 58.08% | 61.39% ± 4.67% | 73.91% | 79.73% | 76.82% ± 4.12% |
| Nasal | 62.51% | 58.42% | 60.47% ± 2.89% | 71.72% | 70.71% | 71.22% ± 0.71% |
| Superior | 53.96% | 52.53% | 53.25% ± 1.01% | 74.11% | 79.10% | 76.61% ± 3.53% |

TABLE 3
Area 3 Summary

| | Day 1 | Day 7 | Average ± SD of | Day 1 | Day 7 | Average ± SD of |
|---|---|---|---|---|---|---|
| Data Set | Vein | Vein | Mean | Tissue | Tissue | Mean |
| Temporal | 67.69% | 53.57% | 60.63% ± 9.98% | 75.19% | 70.14% | 72.67% ± 3.57% |
| Inferior | 64.72% | 65.93% | 65.325% ± 0.86% | 77.28% | 74.74% | 76.01% ± 2.84% |
| Nasal | 64.97% | 67.18% | 66.08% ± 1.56% | 74.91% | 76.09% | 75.5% ± 0.83% |
| Superior | 57.56% | 51.10% | 54.33% ± 4.57% | 77.74% | 81.75% | 79.75% ± 2.84% |

TABLE 4
Area 4 Summary

| | Day 1 | Day 7 | Average ± SD of | Day 1 | Day 7 | Average ± SD of |
|---|---|---|---|---|---|---|
| Data Set | Vein | Vein | Mean | Tissue | Tissue | Mean |
| Temporal | 55.35% | 52.35% | 53.85% ± 2.12% | 69.27% | 78.71% | 73.99% ± 6.68% |
| Inferior | 59.58% | 60.34% | 59.96% ± 0.54% | 70.69% | 75.80% | 73.25% ± 3.61 |
| Nasal | 65.51% | 57.24% | 61.38% ± 5.85% | 74.50% | 73.61% | 74.01% ± 0.63% |
| Superior | 50.56% | 54.49% | 52.53% ± 2.78% | 72.19% | 80.08% | 76.14% ± 5.58% |

TABLE 5
Overall Summary

| Temporal Vein | | Inferior Vein | | Nasal Vein | | Superior Vein | |
|---|---|---|---|---|---|---|---|
| Average | Stan. Dev. | Average | Stan. Dev. | Average | Stan. Dev. | Average | Stan. Dev. |
| 61.86% | 6.92% | 62.80% | 3.73% | 63.37% | 3.97% | 54.19% | 3.09% |
| Temporal Tissue | | Inferior Tissue | | Nasal Tissue | | Superior Tissue | |
| Average | Stan. Dev. | Average | Stan. Dev. | Average | Stan. Dev. | Average | Stan. Dev. |
| 76.67% | 4.35% | 75.55% | 3.33% | 74.17% | 2.25% | 76.74% | 3.81% |

TABLE 6

| | Vein B | Vein B | Artery A | Artery B |
|---|---|---|---|---|
| 522 | 7854.323 | 5434.634 | 12809.989 | 8228.224 |
| 542 | 5629.303 | 3827.825 | 9764.17 | 6440.672 |
| 548 | 5517.069 | 3847.063 | 10742.01 | 6761.768 |
| 560 | 6213.786 | 4629.38 | 12224.08 | 7822.632 |
| 569 | 6419.886 | 4747.695 | 11740.54 | 7952.072 |
| 577 | 5919.917 | 4094.192 | 10447.38 | 6881.608 |
| 586 | 7356.503 | 5177.271 | 12474.99 | 8346.328 |

Embodiments of an imaging system described above are intended to be merely exemplary; numerous variations and modifications will be apparent to those skilled in the art. All such variations and modifications are intended to be within the scope of the present invention. For example, although a relay portion of the discussed embodiments of the invention operates in telecentric configuration, it is recognized that generally a primary objective may operate to produce an intermediate image with finite magnification. In this case, an additional relay lens may be positioned in proximity of the intermediate image. Alternatively or in addition, it is understood that spectral filters may be positioned after the secondary objectives, with respect to the object.

Discussed embodiments and related modified embodiments can be advantageously used not only in medical applications, but also in military applications, agricultural applications such as harvesting, and geology, for example. The implementations of the idea of the invention allow the user to image and characterize dermatological diseases, ocular diseases caused by hypoxia or ischemia, such as glaucoma, optic neuropathies, retinal vascular occlusions (in veins, arteries), retinopathies such as infectious inflammatory or ischemic (e.g. sickle cell disease, retinopathy of prematurity), diabetic retinopathy, macular degeneration, degenerative diseases that ischemia has a role such as Alzheimer's disease, retinal dystrophies or degenerations (e.g. retinitis pigmentosa). Other imaging applications of embodiments such as, for example, imaging of cardiovascular disease or kidney disease with retinal vascular implications, are also contemplated within the scope of the invention. Furthermore, embodiments of the imaging system could be appropriately modified to be used in imaging of other systemic diseases as diagnostic or therapeutic follow up. One non-limiting example would be the use of an embodiment in endoscopic imaging (e.g., in colonoscopy), brain imaging, dermatological imaging where tissues are analyzed for ischemia or response to treatment.

What is claimed is:

1. An apparatus for determining a parameter representing a physiological characteristic of a tissue of a subject, the apparatus comprising:
an optical system including:
an input configured to receive light from the tissue;
an output connected to the input along at least one optical axis;
a spectrally-selective system disposed along the at least one optical axis between said input and output and configured to process the light in a plurality of discrete bandwidths to form a plurality of image-forming beamlets corresponding to said plurality of discrete spectral bandwidths, wherein at least two of said discrete spectral bandwidths correspond to isosbestic wavelengths; and
at least one detector configured to receive the plurality of image-forming beamlets corresponding to said plurality of discrete spectral bandwidths and to form a plurality of images therefrom;
a processor operably connected with the at least one detector; and
a tangible storage medium having computer-readable instructions embedded therein which, when loaded onto the processor, cause the processor
to form a discrete reflectance spectral line defined, from the plurality of images, at wavelengths corresponding to said discrete spectral bandwidths;
to form an isosbestic reflectance spectral line defined, from the plurality of images, at isosbestic wavelengths;
to determine a target value representing an area of spectral graph regions bound by the discrete reflectance spectral line and the isosbestic reflectance spectral line;
to derive the parameter representing a physiological characteristic of the tissue from the determined target value.

2. An apparatus according to claim 1, further comprising
means for relaying an intermediate image of the object along the at least one optical axis, said means for relaying located between the input and the output and having an exit pupil plane; and
wherein said spectrally-selective system includes means for spatially dividing light traversing said means for relaying into multiple light channels, the means for relaying having respectively corresponding entrance pupils that are aligned in said exit pupil plane.

3. An apparatus according to claim 2, further comprising means for imaging said tissue through each of said multiple light channels onto the same detector.

4. An apparatus according to claim 1, wherein said tissue includes an ocular tissue and said physiological characteristic includes an oxygen saturation level of blood in said ocular tissue.

5. An apparatus according to claim 1, wherein the tangible storage medium has computer-readable instructions embedded therein that causes the processor
to calculate an aggregate area of spectral graph regions bound by the discrete reflectance spectral line and the isosbestic reflectance spectral line, wherein the area depends on the physiological characteristic, and
to enable at least one of (i) normalizing said calculated aggregate area by an area under the isosbestic reflectance spectral line, and (ii) normalizing said calculated aggregate area by a coefficient derived based on reflectance values of the discrete reflectance spectral line.

6. An apparatus according to claim 1, wherein the optical system is configured to acquire said plurality of image-forming signals within a time period that is shorter that a duration of a saccade of the subject.

7. A method for determining an oxygen saturation (OS) signature of an ocular tissue of a subject, the method comprising steps of:
a) acquiring, with an optical detector, optical data representing a spectral distribution of light that has been reflected by a plurality of points across a region of interest (ROI) of the ocular tissue, the spectral distribution being defined by a pre-determined number of discrete wavelengths including at least two isosbestic wavelengths;
b) for a point of the plurality of points of the ocular tissue:
determining a first spectral distribution line formed by optical data corresponding to the pre-determined number of discrete wavelengths;
determining a second spectral distribution line formed by optical data corresponding to the at least two isosbestic wavelengths;
determining an aggregate area of spectral graph regions bound by the first and second spectral distribution lines, wherein the aggregate area depends on a level of OS at said point of the plurality of points of the ocular tissue; and
assigning, to said point of the plurality points of the ocular tissue, a value of the determined aggregate area.

8. A method according to claim 7, further comprising c) for the point of the plurality of points of the ocular tissue:
determining a second area under the second spectral distribution line, wherein the second area is independent from the level of OS at said point of the plurality of points of the ocular tissue; and
assigning, to said point of the plurality of points of the ocular tissue, a value of the determined aggregate area that has been divided by the second area.

9. A method according to claim 8, further comprising said assigned value in an array representing a two-dimensional (2D) distribution of the plurality of points across the ROI of the ocular tissue.

10. A method according to claim 8, further comprising steps of:
d) repeating steps b) and c) for each point of the plurality of points of the ocular tissue to assign corresponding values to each of the plurality of said points; and
e) mapping the assigned values into a 2D distribution of the OS signature of blood in the ocular tissue across the ROI.

11. A method according to claim 7, wherein the acquired optical data includes optical data acquired during time period that is shorter than a duration of a saccade of the subject.

12. A method according to claim 7, wherein the determining an aggregate area includes determining an aggregate area of a spectral graph regions bound by said first and second spectral distribution lines, wherein the first spectral distribution line is W-shaped.

13. A method according to claim 7, where the determining an aggregate area includes determining an aggregate area of three spectral graph regions bound by the first and second spectral distribution lines, wherein first and second spectral graph regions of said three spectral graph regions are adjoining at an isosbestic point.

14. A method according to claim 7, further comprising:
relaying an intermediate image of the ocular tissue with a telecentrically-configured optical system to an exit pupil plane of said optical system; and
spatially segmenting the exit pupil plane with a plurality of optical elements having respectively corresponding finite optical powers and entrance pupils that are aligned in said exit pupil plane.

15. A method according to claim 7, wherein the assigning a value to said point of the plurality of points of the ocular tissue includes assigning, to said point of the plurality of points, a value of said determined aggregate area that has been divided by a coefficient calculated based on intensity values corresponding to at least isosbestic points of the first spectral distribution line.

16. A computer program product encoded in a computer-readable medium and usable with a programmable computer processor disposed in a computer system, the computer program product comprising:
computer-readable program code which causes said programmable computer processor to receive data from an optical detector of an optical system, the data representing a discrete spectral distribution of intensity of light reflected by an ocular tissue of a subject and acquired at predetermined wavelengths including at least two isosbestic wavelengths; and
computer-readable program code which causes said programmable computer processor to transform said received data such as to determine an oxygen saturation (OS) value of blood in the ocular tissue of a subject.

17. A computer program product according to claim 16, wherein the acquired data represents a spectral distribution of intensity of light detected within a time period that is shorter than a duration of a saccade of the subject.

18. A computer program product according to claim 16, further comprising computer-readable program code which causes said programmable computer processor to perform at least one of
(i) normalization of said OS value with respect to at least one of the amount of blood in a portion of the ocular tissue that has been imaged, with said optical system, onto the optical detector and the intensity of light that has been detected by said optical detector, and
(ii) displaying a color-coded map of spatial distribution of said OS value across a portion of the ocular tissue that has been imaged through said optical system onto the optical detector.
